(19) United States
(12) Patent Application Publication
(10) Pub. No.: US 2014/0282142 A1
(43) Pub. Date: Sep. 18, 2014

(54) Touch Screen Interface for Imaging System

(71) Applicant: SONOWISE, INC., Cupertino, CA (US)

(72) Inventor: Shengtz Lin, Cupertino, CA (US)

(73) Assignee: SONOWISE, INC., Cupertino, CA (US)

(21) Appl. No.: 13/826,955

(22) Filed: Mar. 14, 2013

(51) Int. Cl. A61B 8/00; G06F 3/0488; G06F 3/0484

(52) U.S. Cl. CPC A61B 8/467; G06F 3/0484; G06F 3/0488
USPC 715/765

(57) ABSTRACT

A system may include a touch screen device configurable to communicate with and control an ultrasound system. The system may further include an ultrasound user interface component configurable from the touch screen device, having one or more customizable properties, and representing an ultrasound system control component. At least one of the customizable properties of the ultrasound user interface component may be associated with a presence of the ultrasound user interface component on the touch screen device. The presence of the ultrasound user interface component on the touch screen device may be configurable via the touch screen device in response to receiving a user selection on the touch screen device.

### BACKGROUND

There may be various challenges associated with the control of imaging systems such as multi-function or multi-purpose ultrasound imaging systems. With different clinical applications, a user interface (UI) layout for an imaging system such as an ultrasound system may be very challenging. For instance, in a clinical application, such as an interventional application (e.g., for anesthesia, operation rooms, needle puncture, etc.), the user interface may be simple, and may only require a few input components (e.g., buttons). However in a cardiac application, the user interface may need to support very complicated input components and knob-operation in order to accomplish examinations and measurements. In obstetrics and gynecology or urology applications, still further or different requirements for the user interface may exist. The user interface system may also need to allow the user to enter a patient name, date of birth, or other patient/clinical related information.

A user interface designer may analyze a clinical application that an imaging system (e.g., ultrasound system) is intended for (e.g., cardiac or vascular), decide the input components (e.g., buttons, keyboard) to be included, and layout the user interface for the imaging system to support that clinical application. Advances in ultrasound systems may increase system complexity and capability and the same ultrasound system may be used in more and more clinical applications, making the user interface design more challenging. The resulting system may need many input components (e.g., keys and buttons) on the user interface area to assure that it covers all possible clinical applications (e.g., cardiac, obstetrics and gynecology, urology, small organ, abdominal, surgery, emergency room, primary care operation, etc.), and this arrangement may confuse a user intending to use the system for a specific examination or application. For instance, many input components (e.g., keys and buttons) designed for cardiac applications may never be used by an obstetrics and gynecology specialist.

### SUMMARY

This application is based in part on the discovery that configurable touch screen systems and interfaces may be used to more flexibly and easily control ultrasound systems intended to be used in multiple clinical applications.

Accordingly, a system may include a touch screen device configurable to communicate with and control an ultrasound system. The system may further include an ultrasound user interface component configurable from the touch screen device, having one or more customizable properties, and representing an ultrasound system control component. At least one of the customizable properties of the ultrasound user interface component may be associated with a presence of the ultrasound user interface component on the touch screen device. The presence of the ultrasound user interface component on the touch screen device may be configurable via the touch screen device in response to receiving a user selection on the touch screen device.

In an embodiment, one or more of the following features may be included. At least one of the customizable properties of the ultrasound user interface component may be associated with a location of the ultrasound user interface component on the touch screen device. The location of the ultrasound user interface component on the touch screen device may be configurable from the touch screen device in response to receiving user input on the touch screen device. A touch screen device processor may be configured to receive a user command from the touch screen device and transmit an ultrasound user interface component parameter based upon, at least in part, the user command, to an ultrasound host processor. A touch screen device memory may be configured to store a library including the ultrasound user interface component, the one or more customizable properties of the ultrasound user interface component, and one or more values associated with the one or more customizable properties of the ultrasound user interface component. The touch screen device may be part of a touch screen panel separate from an image display device of the ultrasound system. The touch screen device may be part of the image display device of the ultrasound system.

In an implementation, one or more of the following features may be included. The ultrasound host processor may be separate from the touch screen device processor. The ultra sound host processor may be configured to receive the ultrasound user interface component parameter and control the ultrasound system based upon, at least in part, the ultrasound user interface component parameter. The ultrasound user interface component may be selected from the group consisting of: a keyboard, a slider, a knob, a paddle, a trackball, and a button. The ultrasound system may include at least one of: a probe transducer, a front-end beam-former, a scan converter, and a signal processor. The ultrasound system may also include an image display device, and scaling of an image displayable on the image display device is configurable from at least one of: the touch screen device and the image display device. The one or more customizable properties of the ultrasound user interface component may include at least one of: shape, size, color, name, orientation, aspect ratio, movement speed, response time, identification number, component type, vertical position, horizontal position, height, width, initial value, foreground color, background color, dimension number, and step value.

A method may include receiving, at one or more processors, a user selection associated with an ultrasound user interface component from a touch screen device configured to communicate with and control an ultrasound system. The method may further include determining, at the one or more processors, whether the ultrasound user interface component is included in a customizable touch screen ultrasound user interface based upon, at least in part, the user selection associated with the ultrasound user interface component. The method may also include, in response to determining that the ultrasound user interface component is included in the customizable touch screen ultrasound user interface, displaying, at a touch screen device display of the touch screen device, the ultrasound user interface component.

In an embodiment, one or more of the following features may be included. The method may include, in response to determining that the ultrasound user interface component is not included in the customizable touch screen ultrasound user interface, displaying the customizable touch screen ultrasound user interface at the touch screen device display of the touch screen device without the ultrasound user interface component. The method may further include determining, at the one or more processors, a location of the ultrasound user interface component in the customizable touch screen ultrasound user interface based upon, at least in part, user input associated with the ultrasound user interface component. The method may also include displaying, at the touch screen device display of the touch screen device, the ultrasound user interface component at the location in the customizable touch screen ultrasound user interface, based upon, at least in part, the user input associated with an ultrasound user interface component.

In an implementation, one or more of the following features may be included. The method may include transmitting an ultrasound user interface component parameter related to a user command associated with the ultrasound user interface component, from a touch screen device processor of the touch screen device, to an ultrasound host processor of the ultrasound system. The method may further include controlling the ultrasound system based upon, at least in part, the user interface component parameter related to the user command associated with the ultrasound user interface component. The method may also include storing, at a touch screen device memory of the touch screen device, one or more values related to the user selection associated with the ultrasound user interface component. The method may additionally include storing, at a touch screen device memory of the touch screen device, a touch screen ultrasound user interface layout corresponding to a user and including one or more values related to the user selection associated with the ultrasound user interface component. Moreover, the method may include, in response to determining the user operating the ultrasound system, displaying the touch screen ultrasound user interface layout corresponding to the user based upon, at least in part, the one or more values related to the user selection associated with the ultrasound user interface component.

A computer program product may reside on a computer readable storage medium having a plurality of instructions stored thereon, which, when executed by a processor, may cause the processor to perform operations. The operations may include receiving, at the processor, a user selection associated with an ultrasound user interface component from a touch screen device configured to communicate with and control an ultrasound system. The operations may further include determining, at the processor, whether the ultrasound user interface component is included in a customizable touch screen ultrasound user interface based upon, at least in part, the user selection associated with the ultrasound user interface component. The operations may also include, in response to determining that the ultrasound user interface component is included in the customizable touch screen ultrasound user interface, displaying, at a touch screen device display of the touch screen device, the ultrasound user interface component.

In an embodiment, one or more of the following features may be included. The operation may include, in response to determining that the ultrasound user interface component is not included in the customizable touch screen ultrasound user interface, displaying the customizable touch screen ultrasound user interface at the touch screen device display of the touch screen device without the ultrasound user interface component. The operations may further include determining, at the processor, a location of the ultrasound user interface component in the customizable touch screen ultrasound user interface, based upon, at least in part, user input associated with the ultrasound user interface component. The operations may also include displaying, at the touch screen device display of the touch screen device, the ultrasound user interface component at the location in the customizable touch screen ultrasound user interface based upon, at least in part, the user input associated with an ultrasound user interface component.

In an implementation, one or more of the following features may be included. The operations may include transmitting an ultrasound user interface component parameter related to a user command associated with the ultrasound user interface component, from a touch screen device processor of the touch screen device, to an ultrasound host processor of the ultrasound system. The operations may include controlling the ultrasound system based upon, at least in part, the user interface component parameter related to the user command associated with the ultrasound user interface component.

### BRIEF DESCRIPTION OF DRAWINGS

The figures are not necessarily to scale, emphasis instead generally being placed upon illustrative principles. The figures are to be considered illustrative in all aspects and are not intended to limit the invention, the scope of which is defined only by the claims.

FIG. 1 is a diagrammatic view of a user interface (UI) layout in accordance with an aspect of the present disclosure;

FIG. 2 is an ultrasound system in accordance with an aspect of the present disclosure;

FIG. 3 is a diagrammatic view of an ultrasound system in accordance with an aspect of the present disclosure;

FIG. 4 is a diagrammatic chart of UI components in accordance with an aspect of the present disclosure;

FIG. 5 is a diagrammatic chart of UI components in accordance with an aspect of the present disclosure;

FIG. 6 is a diagrammatic view of a user interface (UI) layout in accordance with an aspect of the present disclosure;

FIG. 7 is a diagrammatic view of a user interface (UI) layout in accordance with an aspect of the present disclosure; and

FIG. 8 is a flow chart of a method in accordance with an aspect of the present disclosure.

### DETAILED DESCRIPTION

An imaging system such as an ultrasound system may include various components. For example, the ultrasound system may include a probe transducer, which may transform electric signals into mechanical sound waves. The sound waves may be introduced to tissue (i.e., a patient's tissue), and the probe transducer may convert an echo signal received back from the tissue to electric signals. Further, the ultrasound system may include a front-end beam-former that may generate an electric pulse for transducer excitation and may convert the signal into digital format. The front-end beam-former may also provide a delay profile in order to form a beam for both transmitting and receiving, and may also demodulate an amplitude and Doppler signal out of the echo signal. Additionally, the ultrasound system may include a scan converter and a signal processor, which may transform a coordinate for display and may extract the echo amplitude and Doppler signal with, for example, algorithms for gray scale and color flow imaging.

The ultrasound system may also include a user interface (UI) system which may allow the user to set up display modalities and/or control the ultrasound system through the various ultrasound user interface components (e.g., a keyboard, trackball, slide potential meter, knobs, paddle etc.). Moreover, the ultrasound system may additionally include a back end processor, which may perform algorithm calculations. The ultrasound system may also be configured for UI/peripheral interface management and patient file management. The ultrasound system may include other modules or components such as a display (e.g., LCD), speaker, AC/DC power supply, chassis, etc., to form a functional, working imaging system.

Referring now to FIG. 1, a diagrammatic view of fixed UI system 100 of an ultrasound system is shown. Various ultrasound user interface components (e.g., keyboard 102, trackball 104, slide potential 106, knobs 108-116, and paddles 118-126) may be customized moldings. The customized moldings may not be modifiable and may be placed on fixed UI system 100 or a UI area layout with little or no option to rearrange or customize the components molded in the layout. If new components or functions are advanced or introduced, fixed UI system 100 may require redesign in order to fit in a new key or other component. Specialized physicians or other medical professionals or assistants may not be able to eliminate unwanted keys or components not required in their practice. This may leave ultrasound system operation cumbersome or confusing. Example fixed UI system 202 of ultrasound system 200 with a molded or fixed layout 204 is shown in FIG. 2.

Referring now to FIG. 3, in an embodiment of the present disclosure, a customizable UI system 302 may include a touch screen device 304, which may be part of a touch panel 306. As represented in FIG. 3 by a dotted box, touch panel 306 may include a memory (e.g., touch screen device memory 310) and a processor (e.g., touch screen device processor 312), both of which may operate and function in accordance with various memories and processors described herein and/or known to one of skill in the art. Touch screen device 304 may be an input device for ultrasound system 300. Touch screen device 304 may also be configurable to communicate with and control ultrasound system 300. Further, one or more ultrasound user interface (UI) components may be configurable from touch screen device 304. The ultrasound UI components may have one or more customizable properties and may represent ultrasound system control components.

For example, UI system 302 may include a built-in number of ultrasound UI components or primitives that represent ultrasound system control components, such as a trackball, slide potential (slider), knob, button, and/or paddle as discussed above in connection with FIG. 1. UI system 302 may be configured to allow a user to call out the ultrasound UI components, move corresponding ultrasound UI component icons around touch screen device 304, and place the ultrasound UI components where desired on touch screen device 304. In this way, UI system 302 may be configured to allow the user to layout a personal user interface customized by user according to the user's practice or preference.

Further, UI system 302 may be configured to allow the user to customize one or more properties of the ultrasound UI components. One or more of the customizable properties of the ultrasound UI components may be associated with a presence or a location of the ultrasound UI component on touch screen device 304. The customizable properties may include at least one of shape, size, color, name, orientation, aspect ratio, movement speed, response time, identification number, component type, vertical position, horizontal position, height, width, initial value, foreground color, background color, dimension number, and step value.

As discussed above, touch screen device 304 may be part of touch screen panel 306. Touch screen panel 306 may be separate from or in addition to a main image display device 308 or display panel of ultrasound system 300. Main image display device 308 may be a monitor, liquid crystal display (LCD) or other device that allows the user (e.g., a physician or other medical professional) to view gray scale or color flow imaging of a desired area of, for example, a patient. While touch screen device 304 and touch screen panel 306 may be separate from main image display device 308 of ultrasound system 300, in an embodiment, touch screen device 304 may be part of main image display device 308 of ultrasound system 300.

For example, in an embodiment, if ultrasound system 300 includes one panel, touch screen device 304 and main image display device 308 may be combined together. A side or center portion of a panel including main image display device 308 may be allocated for display of an image (e.g., a gray scale and color flow image), and the other side or left and right sides of the center portion may be allocated for the touch screen device.

Upon initiation or power up of ultrasound system 300, touch screen device 304 may load or display a default ultrasound UI layout. Touch screen device 304 may also include and display various ultrasound UI layout options that may already be stored in touch screen device 304 or in ultrasound system 300. The default ultrasound UI layout may include one or more ultrasound UI components (which may be operable touch screen components) such as, for example, a trackball, touch pad, TGC (time gain compensation) slide potential (slider), one or more knobs, one or more paddles to control an ultrasound setting, or one or more buttons for mode triggering. The ultrasound UI components may also include brightness controls and various color settings (e.g., a background color setting), which, when adjusted, may cause touch screen device 304 and/or touch screen panel 306 to appear similar to a UI system (e.g., UI system 100) of an ultrasound system (e.g., ultrasound system 200) with a molded or fixed layout 204, as shown in FIG. 2.

The user (e.g., physician or medical professional) may wish to change the appearance or features of the default ultrasound UI layout. For example, a physician may wish to move a track ball or slider to a different location on the ultrasound UI layout. The user may also wish to remove the track ball or slider from the ultrasound UI layout, or may wish to add a track ball or slider to the ultrasound UI layout.

In an embodiment, the user may remove or add an ultrasound UI component by configuring a customizable property associated with a presence of the component. At least one of the customizable properties of an ultrasound UI component (e.g., a trackball, touch pad, TGC slide pot, knob, paddle, and/or button) may be associated with a presence of the ultrasound UI component on the touch screen device (e.g., touch screen device 304). Referring now also to FIG. 8, the UI system (e.g., UI system 302) may receive 802 a user selection associated with an ultrasound UI component from the touch screen device (e.g., touch screen device 304) configured to communicate with and control an ultrasound system (e.g., ultrasound system 300). The presence of the ultrasound UI component on touch screen device 304 may be configurable via touch screen device 304 in response to receiving the user selection on touch screen device 304.

For example, UI system 302 may allow the user to view a customizable properties menu for a given ultrasound UI component (e.g., a trackball), and the user may, via the menu, choose whether or not the trackball will be present on the ultrasound UI layout. In an implementation, the user may configure the presence of the ultrasound UI component using add/remove features available from UI system 302. In other words, the user may select to add or remove an ultrasound UI component from touch screen device 304. UI system 302 may determine 804 whether the ultrasound UI component is to be included in the customizable touch screen ultrasound UI (i.e., ultrasound UI layout) based upon, at least in part, the user selection associated with the ultrasound UI component (e.g., add/remove selection).

In response to determining that the ultrasound UI component is to be included in the customizable touch screen ultrasound UI (i.e., ultrasound UI layout), the UI system (e.g., UI system 302) may display 806 the ultrasound UI component. The UI system may display the ultrasound UI component at a touch screen device display of the touch screen device (e.g., touch screen device 304). Further, in response to determining that the ultrasound UI component is not to be included in the customizable touch screen ultrasound UI (i.e., ultrasound UI layout), the UI system (e.g., UI system 302) may display 808 the customizable touch screen ultrasound UI (i.e., ultrasound UI layout) without the ultrasound UI component. The UI system (e.g., UI system 302) may display the customizable touch screen ultrasound UI (i.e., ultrasound UI layout) without the ultrasound UI component at the touch screen device display of the touch screen device (e.g., touch screen device 304).

Further, at least one of the customizable properties of the ultrasound UI component may be associated with a location of the ultrasound UI component on the touch screen device (e.g., touch screen device 304). The location of the ultrasound UI component on the touch screen device (e.g., touch screen device 304) may be configurable from the touch screen device in response to receiving user input on the touch screen device. The UI system (e.g., UI system 302) may determine 810 a location of the ultrasound UI component in the customizable touch screen ultrasound UI (i.e., ultrasound UI layout) based upon, at least in part, user input associated with the ultrasound UI component.

For example, the UI system (e.g., UI system 302) may allow the user to select the UI component in response to the user touching the UI component on the touch screen device (e.g., touch screen device 304). Once selected, the UI system (e.g., UI system 302) may allow the user to provide user input associated with a desired location for the ultrasound UI component by allowing the user to drag the UI component to a different location on the touch screen device (e.g., touch screen device 304) with, for example, the user's finger or other touch-capable device such as a stylus or touch pen. The UI system (e.g., UI system 302) may display 812 the ultrasound UI component at the desired location in the customizable touch screen ultrasound UI (i.e., ultrasound UI layout), based upon, at least in part, the user input associated with an ultrasound UI component.

Referring now to FIG. 4, various ultrasound UI component icons (i.e., primitives) are shown. The ultrasound UI component icons (e.g., keyboard 402, trackballs 404-406, slide potential 408, knob 410, paddle 412, and buttons 414-418) shown in FIG. 4 may represent one or more ultrasound UI components (e.g., keyboard 102, trackball 104, slide potential 106, knobs 108-116, and paddles 118-126) shown as customized moldings in fixed layout 204, as previously discussed in connection with FIG. 1. Keyboard 402, trackballs 404-406, slide potential 408, knob 410, paddle 412, and/or buttons 414-418 may be touch responsive icons. In response to a user touching keyboard 402, trackballs 404-406, slide potential 408, knob 410, paddle 412, and/or buttons 414-418, one or more of the touch screen device (e.g., touch screen device 304), UI system (e.g., UI system 302), or ultrasound system (e.g., ultrasound system 300), either individually or in some combination, may call a method or function that may cause the ultrasound system to act or operate as desired.

For example, a keyboard ultrasound UI component (represented by, e.g., keyboard 402) may be a primitive component that may include a set of keys. One or more of the keys on keyboard 402 may have associated values based upon, at least in part, a system setting language. Further a slide potential ultrasound UI component (represented by, e.g., slide potential 408.) may be a primitive component that may slide in two directions. Slide potential 408 may have user-defined minimum and maximum values.

A knob ultrasound UI component (represented by, e.g., knob 410) may be a primitive component that may be rotated clockwise or counterclockwise. Knob 410 may also be pressed. Further, a paddle ultrasound UI component (represented by, e.g., paddle 412) may be a primitive component that may be flicked in two directions. Also, trackball ultrasound UI component (represented by, e.g., trackball 404 or 406) may be a primitive component that may move in all directions. Trackball 404 or 406 may return movement direction and/or speed.

A button ultrasound UI component (represented by, e.g., button 414, 416, or 418) may be a primitive component that may be pressed. Further, a label ultrasound UI component (not shown) may be a primitive component that may include non-interactive text. Additionally, a group ultrasound UI component (not shown) may be a primitive component that may include a non-interactive box that may allow other ultrasound UI components to be grouped together. It should be noted that the ultrasound UI components and/or primitive components discussed herein and shown in the figures (e.g., FIG. 4) are discussed and shown for illustrative purposes only, and other ultrasound UI components and/or primitive components are within the scope of the present disclosure.

Also shown in FIG. 4 are various customizable properties (e.g., name, rotation, size, shape, and/or color) of the ultrasound UI components and/or primitive components. In an embodiment, ultrasound UI components may share a set of customizable properties. The customizable properties may be configured by the user. One or more customizable properties may have no effect, depending on the ultrasound UI component. For example, a slide minimum value may have no effect for a button ultrasound UI component (represented by, e.g., button 414, 416, or 418).

In an embodiment, one or more ultrasound UI components may have a specific set of customizable properties. These ultrasound UI components may have customizable properties unique to the ultrasound UI component type. For example, a rotation step value may be a customizable property unique to a knob ultrasound UI component (represented by, e.g., knob 410).

A unique numeric identification may be a customizable property for an ultrasound UI component. The unique numeric identification may be automatically assigned by the UI system to uniquely identify an ultrasound UI component in a database. This unique numeric identification may not be editable by the user. Further, a component type may be a customizable property for an ultrasound UI component. Component type may be set to any of the ultrasound UI components discussed herein, e.g., button, slider, paddle, etc. Once the component type is configured, the ultrasound UI component may take the figure of the selected component type (e.g., keyboard 402, trackballs 404-406, slide potential 408, knob 410, paddle 412, and buttons 414-418).

Additionally, component name may be a customizable property for an ultrasound UI component. Component name may be set to any text the user desires to describe the ultrasound UI component. For example, as shown in FIG. 4 keyboard 402 may be named “Keyboard 1”). As discussed above and shown in FIG. 4, further customizable properties may be rotation, size, shape, or color. Other customizable properties are discussed below.

A touch screen x-position may be a customizable property for an ultrasound UI component that may represent the horizontal position of the ultrasound UI component on the touch screen device. Further, touch screen y-position may be a customizable property for an ultrasound UI component that may represent the vertical position of the ultrasound UI component on the touch screen device. Touch screen x-position and touch screen y-position may be set numerically via a menu, or may be set by the user through dragging and dropping the ultrasound UI component in the desired location on the touch screen.

A component width may be a customizable property for an ultrasound UI component that may represent how wide the ultrasound UI component will appear on the touch screen device. Further, a component height may be a customizable property for an ultrasound UI component that may represent a height of the ultrasound UI component on the touch screen device. Component width and component height may be set numerically via a menu, or may be set by the user through physically stretching the width or height of the ultrasound UI component on the touch screen device.

A foreground color may be a customizable property for an ultrasound UI component that may represent the foreground color of the ultrasound UI component. Further, background color may be a customizable property for an ultrasound UI component that may represent the background color of the ultrasound UI component. Foreground color and background color may be set via a menu.

Dimension may also be a customizable property for an ultrasound UI component. Dimension may represent whether the ultrasound UI component will be viewed in two dimensions or three dimensions. Dimension may be set via a menu. For example, if “2D” is selected, the ultrasound UI component may be drawn or rendered in two dimensions and may appear two dimensional on the touch screen device (e.g., as shown in FIG. 4). Referring now to FIG. 5, if “3D” is selected, the ultrasound UI component may be drawn or rendered in three dimensions and may appear three dimensional on the touch screen device. For example, and as shown in FIG. 5, trackball 502, buttons 504-506, slide potential 508, and/or keyboard 510 may be appear three dimensional on the touch screen device if a dimension customizable property for corresponding ultrasound UI components is set for three dimensions.

Additionally, descriptive text (e.g., name as shown in FIG. 4 and FIG. 5) may be a customizable property for an ultrasound UI component that may represent a name or title of the ultrasound UI component. Descriptive text may be entered via a menu. Further, descriptive text x-position and descriptive text y-position may be customizable properties for an ultrasound UI component that may represent a horizontal position for the descriptive text on the touch screen device, and a vertical position for the descriptive text on the touch screen device, respectively. Descriptive text x-position and descriptive text y-position may be set numerically via a menu, or may be set by the user through dragging and dropping the descriptive text of the ultrasound UI component in the desired location on the touch screen.

Component orientation may also be a customizable property for an ultrasound UI component that may represent the orientation with which the ultrasound UI component will appear on the touch screen device. For example, component orientation may be set to ninety degrees, and the ultrasound UI component may appear on the touch screen device rotated ninety degrees. Component orientation may be set numerically, or may be set by the user through rotating (e.g., by finger, stylus, touch pen, etc.) the ultrasound UI component to the desired orientation on the touch screen device.

In an embodiment, one or more customizable properties for an ultrasound UI component may be associated with values for the ultrasound UI component. For example, a slider ultrasound UI component may have an initial value, a slide step value, a slide minimum value, and a slide maximum value. Further, a knob ultrasound UI component may have a rotation step value. A trackball ultrasound UI component may have a motion step value. A paddle ultrasound UI component may have a flick step value. The slide step value, rotation step value, motion step value, and flick step values may indicate the effect of user's slide, rotation, motion, and flick (input via the touch screen device) on the UI system. These customizable properties may be set numerically and may have default values.

Other customizable properties for an ultrasound UI component may be associated with sound, or another effect that may occur, when the ultrasound UI component is activated. For example, a click customizable property, if set, may cause the UI system to make a click sound when the corresponding ultrasound UI component is activated.

In an embodiment, the UI system may include touch screen device processor 312. The touch screen device processor 312 may be configured to receive a user command from the touch screen device (e.g., touch screen device 304). Touch screen device processor 312 may be further configured to transmit an ultrasound UI component parameter based upon, at least in part, the user command, to an ultrasound host processor (located, e.g., in ultrasound system 300). In this way, the UI system (e.g., UI system 302) may transmit 814 the UI component parameter related to a user command associated with the ultrasound UI component, from touch screen device processor 312 of touch screen device 304, to an ultrasound host processor of the ultrasound system (e.g., ultrasound system 300).

The ultrasound host processor may be separate from touch screen device processor 312. Further, the ultrasound host processor may be configured to receive the ultrasound UI component parameter. Additionally, the ultrasound host processor may be configured to control the ultrasound system based upon, at least in part, the ultrasound UI component parameter. In this way, the UI system may control 816 the ultrasound system based upon, at least in part, the UI component parameter related to the user command associated with the ultrasound UI component.

For example, on touch screen device 304, if a key is pressed or the slide potential level is shifted (e.g., with the user's finger, stylus, touch pen, etc.), touch screen device processor 312 may receive input and may transmit a serial of parameters according to a UI command protocol. The ultrasound host processor may decode the parameters and may issue a command to the ultrasound system. The ultrasound host processor may also collect a status from the ultrasound system and transmit the status to touch screen device 304 or the main image display 308.

Referring back to FIG. 3, in an embodiment, the UI system may include touch screen device memory 310. The UI system may store 818 one or more values related to a user selection associated with an ultrasound UI component at touch screen device memory 310. Touch screen memory 310 may be configured to store or include a library. The library may store or include various ultrasound UI components. As shown in FIG. 4, the library may also store or include one or more customizable properties (e.g., name, rotation, size, shape and/or color) of the ultrasound UI components. Also as shown in FIG. 4, the library may additionally include and/or store one or more values associated with the one or more customizable properties of the ultrasound UI components.

The ultrasound UI components, customizable properties, and values shown in FIG. 4 and discussed herein are shown and discussed for illustrative purposes only, and other possible ultrasound UI components, customizable properties, and values not shown in FIG. 4 are within the scope of the present disclosure. For example, the customizable properties may further include orientation and aspect ratio.

Referring now to FIG. 6 and FIG. 7, examples of customized ultrasound UI layouts (e.g., customized ultrasound UI layouts 602 and 702) in accordance with the present disclosure are shown. As shown in FIG. 6, customized ultrasound UI layout 602 may include one or more ultrasound UI component icons representing, e.g., keyboard 604, slide potential 606, buttons 608-614, and trackball 616. As shown in FIG. 7, customized ultrasound UI layout 702 may include one or more ultrasound UI component icons representing, e.g., keyboard 704, slide potential 706, buttons 708-714, and trackball 716. Also as shown in FIG. 7, the color of each ultrasound UI component may be a customizable property, and the user may set the color such that, for example, button 708 appears yellow and button 714 appears purple. In this way, users may configure and/or customize ultrasound UI layouts (e.g., customized ultrasound UI layouts 602 and 702) to adapt to different clinical applications and/or personal preference.

A user may name and save the customized ultrasound UI layout (e.g., customized ultrasound UI layouts 602 and 702) in the UI system. The UI system may store 820, at the touch screen device memory of the touch screen device, a touch screen ultrasound UI layout corresponding to the user (e.g., customized ultrasound UI layouts 602 and 702). The touch screen ultrasound UI layout corresponding to the user may include one or more values related to a user selection associated with an ultrasound user interface component. Further, the user may call the saved, customized ultrasound UI layout (e.g., customized ultrasound UI layouts 602 and 702) when subsequently using the ultrasound system. For example, in response to determining 822 what user is operating the ultrasound system, the UI system may display 824 the touch screen ultrasound UI layout corresponding to that user based upon, at least in part, one or more values related to a user selection associated with an ultrasound UI component.

This customized ultrasound UI layout may make ultrasound system operation more clear and may make it easier to conduct a specific exam. In this way, the customizable ultrasound UI may be more flexible, and may be more suitable for various clinical applications. The UI system may also have various default ultrasound user UI layouts for different clinical examinations.

In an embodiment, the ultrasound UI components may have descriptive text in different languages. Further, a user may select a language from a setting and the UI system may adjust associated text accordingly such that speakers of the language may operate the UI system.

In an embodiment the only physical buttons or other fixed components required in the UI system may be a power on/off and a reset or standby button or switch. In this way, the customizable ultrasound UI on the touch screen device may be easier to clean and sanitize, especially in a hospital or other clinical setting.

Further, an ultrasound UI component (e.g., a keyboard, trackball, slide potential meter, knob, paddle, button, etc.) may have an associated set of tunable, customizable, and/or configurable settings or parameters. For example, a trackball control element (i.e., ultrasound UI component) as shown in FIG. 6 may be configurable such that its movement speed or speed along a particular axis such as a positive x-axis or negative y-axis can be adjusted.

Scaling of an image displayable on the main image display device (e.g., main image display device 308) may configurable from the touch screen device. Further, scaling of an image displayable on the main image display device may configurable from the main image display device. For example, the legend and/or scaling of image data may be configurable such that units of measure on the main image display may be adjusted by menu or by touch.

An ultrasound UI component may have one or more associated event-driven methods. These methods may be invoked in response to the user initiating an action on the ultrasound UI component. The methods may have a function pointer which may point to a fixed system function. Initially, the function pointer may not be set (e.g., may have a NULL value). The function pointer may be set by the user.

For example, a keyboard ultrasound UI component may have associated KeyPressed( ) and KeyReleased( ) methods, which may be invoked in response to the user pressing a key, and releasing a key, respectively. Further, a slider ultrasound UI component may have an associated SliderMoved( ) method. This method may receive a motion value for the slider in response to the user sliding the slider. Additionally, a knob ultrasound UI component may have an associated KnobRotated( ) method. This method may receive a positive (clockwise) or negative (counterclockwise) value representing the amount of rotation, in response to the user rotating the knob.

In an embodiment, a paddle ultrasound UI component may have an associated PaddleFlicked( ) method. This method may receive a positive or negative value in response to the user flicking the paddle. Further, a trackball ultrasound UI component may have an associated TrackballMoved( ) method. This method may receive a value corresponding to motion along the x-axis and a value corresponding to motion along the y-axis, in response to the user moving the trackball. Depending on the design of the UI system and/or touch screen device, the values may be combined using an OR function or operator to form a larger structure (e.g. two 8-bit values may form a 16-bit value). In this way, a single value may be formed and passed to the method. Additionally a button ultrasound UI component may have associated ButtonPressed( ) and ButtonReleased( ) methods, which may be invoked in response to the user pressing a button, and releasing a button, respectively.

The UI system may also include fixed functions which may be called by one or more of the event-driven methods associated with the ultrasound UI component. For example, the functions ValueGain( ), ValueAngle( ), and ValueVolume( ) may change variable values. These functions may allow the corresponding values of a system variable (e.g., gain, angle, volume) to be set. These functions may check to ensure the corresponding value is within bounds. Further, the functions StepSectorWidth( ), StepLineDensity( ), StepFocus( ), StepDepth( ), and StepBaseline( ) may change variables with fixed values. These system variables (e.g., sector width, lines density, focus, depth, and baseline) may have fixed values. The corresponding functions may allow the value to be set to the previous or next fixed value. If the function is passed a positive value, the corresponding variable's value may be set to the next fixed value. If the function is passed a negative value, the corresponding variable's value may be set to the previous fixed value.

Also, the functions MeasureDistance( ), MeasureEllipse( ), and MeasureTrace( ), MeasureVolume( ) may begin or end a corresponding measurement mode. The functions ModeB( ), ModePW( ), ModeCFM( ), ModeM( ), ModeTHI( ), and ModeTDI( ) may change modes during a real-time scan.

Additionally, the functions KeyFreeze( ), KeyExam( ), KeyPatient( ), KeyDelete( ), KeyMenu( ), KeyReport( ), KeyPrint( ), KeySave( ), KeyEndExam( ), KeyAnnotation( ), and KeyBodyMark( ) may invoke common actions or functions (e.g., freeze, exam, patient, delete, menu, report, print, save, end exam, annotation, body mark) found on ultrasound systems. It should be noted that the methods and functions discussed herein are describe for illustrative purpose only, and methods and functions associated with different features common to ultrasound systems or associated with the same features but different names common to ultrasound systems are within the scope of the present disclosure.

During an ultrasound UI layout editing mode, the user may add a new ultrasound UI component or edit an existing ultrasound UI component. An ultrasound UI layout may be saved as a profile. Initially, all components may be displayed. The following commented code, provided for illustrative purposes only, may be used, in part, to display the components:

// loop through all components in the selected profile's
database
for (int i = 0; i < Profile.NumComponents; i++)
{
// draw the component based on the component's (x,y)
position
Profile.Component[i].Draw( );
}

In an embodiment, regardless of whether the user selects a new component, or selects an existing component, the ultrasound UI component properties may be displayed, and the user may edit them. When a component property is edited, the value may be saved to the component. The following commented code, provided for illustrative purposes only, may be used, in part, to save the component value:

// example of setting the x position of component
// where value is a number entered by the user
void Component::SetPositionX(int value)
{

// check if new value is inside touch panel boundaries
if ((value >= 0) && (value < TouchPanelWidth))
{

positionX = value;

}

}
// example of setting the descriptive text of component
// where text is a string entered by the user
void Component::SetDescriptiveText(const char *text)
{

// copy new text to variable descriptiveText, making
// sure not to go past memory bounds
strcpy(descriptiveText, text, sizeof(descriptiveText)−1));

}

In an embodiment, the user may also edit the ultrasound UI component's event-driven methods and may set a fixed system function call to be invoked. The following commented code, provided for illustrative purposes only, may be used, in part, to set a fixed system function call to be invoked:

// example of function call if user sets ButtonPressed( )
method
// to invoke system function KeyFreeze( )
myButton−>SetButtonPressed(&KeyFreeze);
// example of setting function pointer used inside
ButtonPressed( )
// method. This function is called when the user edits the
function
// invoked by the ButtonPressed( ) method.
void Component::SetButtonPressed(void
(*SystemFunctionPtr)(int))
{

buttonPressedFunc = SystemFunctionPtr;

}
// example of ButtonPressed( ) method, not editable by user.
This
// function is called when user “presses” the button on the
touchpanel.
// if the buttonPressedFunc had been previously set to
KeyFreeze( ),
// the KeyFreeze( ) function will be called and handled by
the system.
void Component::ButtonPressed(int value)
{

if (buttonPressedFunc != NULL)
{

(*buttonPressedFunc)(value);

}
// other code here to handle redrawing of button on screen,

etc.
}

While using the touch screen device, the user may perform different actions on the touch screen device, such as pressing down on the touch screen device, or moving their finger across the touch screen device. The UI system may determine a corresponding action, and may call a corresponding event-driven method. The following commented code, provided for illustrative purposes only, may be used, in part, to call an event driven method:

// example of user pressing the screen at position (s,t)
// loop through the component list
for (int i = 0; i < Profile.NumComponents; i++)
{

// get component boundaries
Xmin = Profile.Component[i].GetXPosition( );
Ymin = Profile.Component[i].GetYPosition( );
Xmax = Xmin + Profile.Component[i].GetWidth( );
Ymax = Ymin + Profile.Component[i].GetHeight( );
// check if the interaction position (s,t) is within

component bounds

if ((s >= Xmin) && (s < Xmax) && (t >= Ymin) && (t <

Ymax))

{

// within bounds, invoke event-driven method
// based on component type
switch (Profile.Component[i].GetComponentType( ))
{

// if button, call ButtonPressed( )
case Component::Button:

Profile.Component[i].ButtonPressed( );

break;

// if knob, call KnobPressed( )
case Component::Knob:

Profile.Component[i].KnobPressed( );

break;

default:

break;

}

}

}
// example of user moving across the screen with start
position (s,t)
// and motion values (u,v), where u is motion along the x-
axis, and
// v is motion along the y-axis
// loop through the component list
for (int i = 0; i < Profile.NumComponents; i++)
{

// get component boundaries
Xmin = Profile.Component[i].GetXPosition( );
Ymin = Profile.Component[i].GetYPosition( );
Xmax = Xmin + Profile.Component[i].GetWidth( );
Ymax = Ymin + Profile.Component[i].GetHeight( );
// check if the interaction position (s,t) is within

component bounds

if ((s >= Xmin) && (s < Xmax) && (t >= Ymin) && (t <

Ymax))

{

// within bounds, invoke event-driven method
// based on component type
switch (Profile.Component[i].GetComponentType( ))
{

case Component::Slider:

// check if slider is horizontal, or vertical
if (Profile.Component[i].GetOrientation( ) ==

Orientation::Horizontal)

{

// pass it the motion value along the x-axis
Profile.Component[i].SliderMoved(u);

}
else
{

// pass it the motion value along the y-axis
Profile.Component[i].SliderMoved(v);

}
break;

case Component::Knob:

// calculate the amount of rotation based on u,v
// and pass to KnobRotated( );
int rotation = CalcRotation(u,v);
Profile.Component[i].KnobRotated(rotation);
break;

default:

break;

}

}

}

In one embodiment, the invention relates to a configurable user interface suitable for controlling, designing or reconfiguring a user interface layout and input element's function using drag and drop graphical elements such as icons that are touch screen movable and lockable elements that can be assembled like building blocks to generate a touch screen user interface. Each user assembled or configured touch screen interface can be locked and saved by the user in one embodiment. In one embodiment, the component library is stored in the user interface processor.

The present disclosure discusses embodiments in the context of ultrasound imaging systems, however, these embodiments are not intended to be limiting and those skilled in the art will appreciate that the present disclosure can also be applied to other imaging systems.

The aspects, embodiments, features, and examples of the present disclosure are to be considered illustrative in all respects and are not intended to limit the invention, the scope of which is defined only by the claims. Other embodiments, modifications, and usages will be apparent to those skilled in the art without departing from the spirit and scope of the present disclosure.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the present disclosure; each section can apply to any aspect, embodiment, or feature of the present disclosure.

Throughout the application, where compositions are described as having, including, or comprising specific components, or where processes are described as having, including or comprising specific process steps, it is contemplated that compositions of the present teachings also consist essentially of, or consist of, the recited components, and that the processes of the present teachings also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components. Further, it should be understood that elements and/or features of a system, a composition, an apparatus, or a method described herein can be combined in a variety of ways without departing from the spirit and scope of the present teachings, whether explicit or implicit herein.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise. In addition, where the use of the term “about” is before a quantitative value, the present teachings also include the specific quantitative value itself, unless specifically stated otherwise.

It should be understood that the order of steps or order for performing certain actions or operations is immaterial so long as the present teachings remain operable. Moreover, two or more steps or actions or operations may be conducted simultaneously.

Where a range or list of values is provided, each intervening value between the upper and lower limits of that range or list of values is individually contemplated and is encompassed within the invention as if each value were specifically enumerated herein. In addition, smaller ranges between and including the upper and lower limits of a given range are contemplated and encompassed within the invention. The listing of exemplary values or ranges is not a disclaimer of other values or ranges between and including the upper and lower limits of a given range.

The present disclosure may be embodied in many different forms, including, but in no way limited to, computer program logic for use with a processor (e.g., a microprocessor, microcontroller, digital signal processor, or general purpose computer), programmable logic for use with a programmable logic device, (e.g., a Field Programmable Gate Array (FPGA) or other PLD), discrete components, integrated circuitry (e.g., an Application Specific Integrated Circuit (ASIC)), or any other means including any combination thereof. In one embodiment of the present invention, some or all of the processing of the data used to generate a control signal or initiate a user interface command is implemented as a set of computer program instructions that is converted into a computer executable form, stored as such in a computer readable medium, and executed by a microprocessor under the control of an operating system. In one embodiment, output control signals from a controller are transformed into processor understandable instructions suitable for responding to stylus, finger or other user inputs, controlling a graphical user interface, control and graphic signal processing, scaling a legend of an ultrasound image using a touch screen, touch screen sliding and dragging, other data as part of a graphic user interface and other features and embodiments as described above.

Computer program logic implementing all or part of the functionality previously described herein may be embodied in various forms, including, but in no way limited to, a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. For example, a computer program product may reside on a computer readable storage medium having a plurality of instructions stored thereon, which, when executed by a processor, cause the processor to perform operations discussed herein.

The computer program may be fixed in any form in a signal that is transmittable to a computer using any of various communication technologies, including, but in no way limited to, analog technologies, digital technologies, optical technologies, wireless technologies, networking technologies, and internetworking technologies. The computer program may be distributed in any form as a removable storage medium with accompanying printed or electronic documentation (e.g., shrink-wrapped software), preloaded with a computer system (e.g., on system ROM or fixed disk), or distributed over a network.

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink-wrapped software), preloaded with a computer system (e.g., on system ROM or fixed disk), or distributed from a server or electronic bulletin board over the communication system (e.g., the Internet or World Wide Web).

Various examples of suitable processing modules are discussed below in more detail. As used herein a module refers to software, hardware, or firmware suitable for performing a specific data processing or data transmission task. Typically, in a preferred embodiment a module refers to a software routine, program, or other memory resident application suitable for receiving, transforming, routing and processing instructions, or various types of data such as ultrasound modes, color modes, ultrasound mammography data, ultrasound infant or prenatal data, ultrasound cardiac data, icons, touch screen primitives, and other information of interest.

Computers and computer systems described herein may include an operatively associated machine-readable medium such as computer-readable media such as memory for storing software applications used in obtaining, processing, storing and/or communicating data. It can be appreciated that such memory can be internal, external, remote or local with respect to its operatively associated computer or computer system.

The term “machine-readable medium” includes any medium that is capable of storing, encoding or carrying a set of instructions for execution by the machine and that cause the machine to perform any one or more of the methodologies of the present disclosure. While the machine-readable medium is shown in an example embodiment to be a single medium, the term “machine-readable medium” should be taken to include a single medium or multiple media (e.g., a database, one or more centralized or distributed databases and/or associated caches and servers) that store the one or more sets of instructions.

Memory may also include any means for storing software or other instructions including, for example and without limitation, a hard disk, an optical disk, floppy disk, DVD (digital versatile disc), CD (compact disc), memory stick, flash memory, ROM (read only memory), RAM (random access memory), DRAM (dynamic random access memory), PROM (programmable ROM), EEPROM (extended erasable PROM), and/or other like computer-readable media.

In general, computer-readable memory media applied in association with embodiments of the invention described herein may include any memory medium capable of storing instructions executed by a programmable apparatus. Where applicable, method steps described herein may be embodied or executed as instructions stored on a computer-readable memory medium or memory media.

It is to be understood that the figures and descriptions of the invention have been simplified to illustrate elements that are relevant for a clear understanding of the present disclosure, while eliminating, for purposes of clarity, other elements. Those of ordinary skill in the art will recognize, however, that these and other elements may be desirable. However, because such elements are well known in the art, and because they do not facilitate a better understanding of the present disclosure, a discussion of such elements is not provided herein. It should be appreciated that the figures are presented for illustrative purposes and not as construction drawings. Omitted details and modifications or alternative embodiments are within the purview of persons of ordinary skill in the art.

It can be appreciated that, in certain aspects of the present disclosure, a single component may be replaced by multiple components, and multiple components may be replaced by a single component, to provide an element or structure or to perform a given function or functions. Except where such substitution would not be operative to practice certain embodiments of the present disclosure, such substitution is considered within the scope of the present disclosure.

The examples presented herein are intended to illustrate potential and specific implementations of the present disclosure. It can be appreciated that the examples are intended primarily for purposes of illustration of the present disclosure for those skilled in the art. There may be variations to these diagrams or the operations described herein without departing from the spirit of the present disclosure. For instance, in certain cases, method steps or operations may be performed or executed in differing order, or operations may be added, deleted or modified.

Furthermore, whereas particular embodiments of the present disclosure have been described herein for the purpose of illustrating the present disclosure and not for the purpose of limiting the same, it will be appreciated by those of ordinary skill in the art that numerous variations of the details, materials and arrangement of elements, steps, structures, and/or parts may be made within the principle and scope of the present disclosure without departing from the invention as described in the claims.

What is claimed is:

1. A system comprising:
a touch screen device configurable to communicate with and control an ultrasound system;
an ultrasound user interface component configurable from the touch screen device, having one or more customizable properties, and representing an ultrasound system control component;
at least one of the customizable properties of the ultrasound user interface component being associated with a presence of the ultrasound user interface component on the touch screen device; and
the presence of the ultrasound user interface component on the touch screen device being configurable via the touch screen device in response to receiving a user selection on the touch screen device.

2. The system of claim 1, further comprising:
at least one of the customizable properties of the ultrasound user interface component being associated with a location of the ultrasound user interface component on the touch screen device; and
the location of the ultrasound user interface component on the touch screen device being configurable from the touch screen device in response to receiving user input on the touch screen device.

3. The system of claim 1, further comprising:
a touch screen device processor configured to receive a user command from the touch screen device and transmit an ultrasound user interface component parameter based upon, at least in part, the user command, to an ultrasound host processor.

4. The system of claim 1, further comprising:
a touch screen device memory configured to store a library including the ultrasound user interface component, the one or more customizable properties of the ultrasound user interface component, and one or more values associated with the one or more customizable properties of the ultrasound user interface component.

5. The system of claim 1, wherein the touch screen device is part of a touch screen panel separate from an image display device of the ultrasound system.

6. The system of claim 1, wherein the touch screen device is part of the image display device of the ultrasound system.

7. The system of claim 3, further comprising:
the ultrasound host processor being separate from the touch screen device processor, the ultra sound host processor configured to receive the ultrasound user interface component parameter and control the ultrasound system based upon, at least in part, the ultrasound user interface component parameter.

8. The system of claim 1, wherein the ultrasound user interface component is selected from the group consisting of: a keyboard, a slider, a knob, a paddle, a trackball, and a button.

9. The system of claim 1, wherein the ultrasound system includes at least one of: a probe transducer, a front-end beam-former, a scan converter, and a signal processor.

10. The system of claim 1, wherein the ultrasound system includes an image display device, and scaling of an image displayable on the image display device is configurable from at least one of: the touch screen device and the image display device.

11. The system of claim 1, wherein the one or more customizable properties of the ultrasound user interface component include at least one of: shape, size, color, name, orientation, aspect ratio, movement speed, response time, identification number, component type, vertical position, horizontal position, height, width, initial value, foreground color, background color, dimension number, and step value.

12. A method comprising:
receiving, at one or more processors, a user selection associated with an ultrasound user interface component from a touch screen device configured to communicate with and control an ultrasound system;
determining, at the one or more processors, whether the ultrasound user interface component is included in a customizable touch screen ultrasound user interface based upon, at least in part, the user selection associated with the ultrasound user interface component; and
in response to determining that the ultrasound user interface component is included in the customizable touch screen ultrasound user interface, displaying, at a touch screen device display of the touch screen device, the ultrasound user interface component.

13. The method of claim 12, further comprising:
in response to determining that the ultrasound user interface component is not included in the customizable touch screen ultrasound user interface, displaying the customizable touch screen ultrasound user interface at the touch screen device display of the touch screen device without the ultrasound user interface component.

14. The method of claim 12, further comprising:
determining, at the one or more processors, a location of the ultrasound user interface component in the customizable touch screen ultrasound user interface based upon, at least in part, user input associated with the ultrasound user interface component; and
displaying, at the touch screen device display of the touch screen device, the ultrasound user interface component at the location in the customizable touch screen ultrasound user interface, based upon, at least in part, the user input associated with an ultrasound user interface component.

15. The method of claim 12, further comprising:
transmitting an ultrasound user interface component parameter related to a user command associated with the ultrasound user interface component, from a touch screen device processor of the touch screen device, to an ultrasound host processor of the ultrasound system.

16. The method of claim 15, further comprising:
controlling the ultrasound system based upon, at least in part, the user interface component parameter related to the user command associated with the ultrasound user interface component.

17. The method of claim 12, further comprising:
storing, at a touch screen device memory of the touch screen device, one or more values related to the user selection associated with the ultrasound user interface component.

18. The method of claim 12, further comprising:
storing, at a touch screen device memory of the touch screen device, a touch screen ultrasound user interface layout corresponding to a user and including one or more values related to the user selection associated with the ultrasound user interface component.

19. The method of claim 18, further comprising:
in response to determining the user operating the ultrasound system, displaying the touch screen ultrasound user interface layout corresponding to the user based upon, at least in part, the one or more values related to the user selection associated with the ultrasound user interface component.

20. A computer program product residing on a computer readable storage medium having a plurality of instructions stored thereon, which, when executed by a processor, cause the processor to perform operations comprising:
receiving, at the processor, a user selection associated with an ultrasound user interface component from a touch screen device configured to communicate with and control an ultrasound system;
determining, at the processor, whether the ultrasound user interface component is included in a customizable touch screen ultrasound user interface based upon, at least in part, the user selection associated with the ultrasound user interface component; and
in response to determining that the ultrasound user interface component is included in the customizable touch screen ultrasound user interface, displaying, at a touch screen device display of the touch screen device, the ultrasound user interface component.

21. The computer program product of claim 20, wherein the operations further comprise:
in response to determining that the ultrasound user interface component is not included in the customizable touch screen ultrasound user interface, displaying the customizable touch screen ultrasound user interface at the touch screen device display of the touch screen device without the ultrasound user interface component.

22. The computer program product of claim 20, wherein the operations further comprise:
determining, at the processor, a location of the ultrasound user interface component in the customizable touch screen ultrasound user interface, based upon, at least in part, user input associated with the ultrasound user interface component; and
displaying, at the touch screen device display of the touch screen device, the ultrasound user interface component at the location in the customizable touch screen ultrasound user interface based upon, at least in part, the user input associated with an ultrasound user interface component.

23. The computer program product of claim 20, wherein the operations further comprise:
transmitting an ultrasound user interface component parameter related to a user command associated with the ultrasound user interface component, from a touch screen device processor of the touch screen device, to an ultrasound host processor of the ultrasound system.

24. The computer program product of claim 20, wherein the operations further comprise:
controlling the ultrasound system based upon, at least in part, the user interface component parameter related to the user command associated with the ultrasound user interface component.
